(19) United States
(12) Patent Application Publication
(10) Pub. No.: US 2024/0139407 A1
(43) Pub. Date: May 2, 2024

(54) TUBE CONNECTOR FOR AN INFUSION TUBE, INFUSION SET, AND METHOD FOR VENTING AN INFUSION TUBE

(71) Applicant: B. Braun Melsungen AG, Melsungen (DE)

(72) Inventors: Karl Martin Berg, Melsungen (DE); Florin Kopp, Schortens (DE)

(21) Appl. No.: 18/282,314

(22) PCT Filed: Mar. 15, 2022
(86) PCT No.: PCT/EP2022/056639

§ 371 Date: Sep. 15, 2023

(30) Foreign Application Priority Data
Mar. 17, 2021 (DE) 10 2021 202 578.1

(51) Int. Cl. A61M 5/14; A61M 5/162

(52) U.S. Cl. CPC A61M 5/1413; A61M 5/162; A61M 2005/1402; A61M 2005/1623; A61M 2205/128

(57) ABSTRACT

A tube connector for an infusion tube is connectable to an infusion container. The tube connector has a first fluid opening that is connectable to the infusion tube or to a drip chamber connected to the infusion tube to provide a first fluid connection between the infusion container and the infusion tube. The tube connector has a second fluid opening connectable to the infusion tube to provide a second fluid connection between the infusion container and the infusion tube. The tube connector has a blocking device configured to block a fluid connection between the infusion tube connected to the second fluid opening and the infusion container connected to the tube connector to prevent a release of fluid from the infusion container into the infusion tube.

### CROSS-REFERENCE TO RELATED APPLICATIONS

This application is the United States national stage entry of International Application No. PCT/EP2022/056639, filed on Mar. 15, 2022, and claims priority to German Application No. 10 2021 202 578.1, filed on Mar. 17, 2021. The contents of International Application No. PCT/EP2022/056639 and German Application No. 10 2021 202 578.1 are incorporated by reference herein in their entireties.

### FIELD

The invention relates to a tube connector for an infusion tube, an infusion set, and alternative methods for venting an infusion tube.

### BACKGROUND

Many substances that are administered to patients as infusions or in a comparable form of delivery have a considerable hazard potential outside the actual therapeutic application. Such substances are referred to below as “toxic”. Examples include drugs with carcinogenic, mutagenic and reprotoxic properties (so-called “CMR drugs”) or sensitizing drugs. In cancer therapy, drugs are used which lead to damage of growth-intensive tumor cells, but which also have a carcinogenic effect due to their mechanism of action and are therefore to be classified as CMR drugs. Contact of non-therapeutic persons with toxic substances must be avoided. Closed system transfer devices (CSTDs) and delivery systems that prevent inadvertent contact with the drugs are therefore frequently used for the manufacture and administration of drugs containing toxic substances.

An important component of such systems are couplings that provide a tight fluid connection between the individual components of the system (containers, fittings, ports, tubes, etc.) and ideally dry-lock after the connection is disconnected. Such couplings typically consist of a first coupling device attached to the end of one of the components of the system to be coupled together, and a second coupling device complementary to the first coupling device attached to the end of the other of the components to be coupled together. The first and second coupling devices are connected to each other, for example, by being inserted into each other or otherwise engaged with each other to establish a fluid connection between the components of the system to be coupled. This process is also referred to as “connecting” the coupling. A coupling is referred to as “detachable” if the interconnected coupling devices can be disconnected from each other in a non-destructive manner to reconnect the fluid connection between the two components of the system. This process is also referred to as “disconnecting” the coupling.

A coupling is described as “dry-locking” if no significant quantities of fluid can escape from the components of the system when the coupling is disconnected and thereafter, and the coupling devices are not wetted with fluid on their external surfaces. This prevents the environment from contamination, for example, by leakage or dripping on the surfaces of the coupling devices. Such a coupling is usually referred to as a “dry coupling”, “dry locking coupling”, “dry-break coupling”, “dry-connect coupling”, “dry connection”, or “closed connection”, or is associated with the term “automatic self-sealing technology”.

A preferred dry-locking coupling is a so-called needle-based dry-locking coupling. This comprises a coupling device with a hollow needle and a first septum and a coupling device with a second septum. When the coupling is connected, the needle first pierces the first septum and then the second septum. When disconnecting, the needle is pulled out of the second septum and then retracted behind the first septum. Then the first septum and the second septum dry-locks the respective coupling device. A needle-based dry-locking coupling device is described in German Patent Application No. 10 2019 217 984.3.

Another well-known coupling of this type is the mandrel-countermandrel coupling. This comprises a mandrel coupling element as a first coupling device and a counter mandrel coupling element as a complementary second coupling device. To establish a coupling state, a mandrel of the mandrel coupling element moves a counter mandrel of the counter mandrel coupling element against a spring force into a counter mandrel receptacle so that the opening of the mandrel projects into the counter mandrel receptacle and, together with the counter mandrel, forms a fluid channel that runs through the counter mandrel receptacle.

Dry-locking couplings are usually not symmetrical in construction, i.e. the first coupling device and the second coupling device are not identical in construction but different from each other and complementary in shape. Couplings that are not dry-locking may also have a non-symmetrical construction. An example of non-symmetrically constructed coupling devices that are complementary to each other are the coupling devices of the needle-based dry-locking coupling described above.

Generally, non-symmetrically constructed complementary coupling devices of a coupling are hereinafter referred to as type A coupling device and type B coupling device, where one type A coupling device and one type B coupling device may form a coupling, but not two type A coupling devices or two type B coupling devices.

Furthermore, systems for the preparation and administration of drugs are often equipped with a pressure equalization mechanism so that, for example, there is no unintentional leakage of the drug when fluids are injected into or removed from the drug vessel.

An infusion set (also referred to as “infusion kit” or “infusion system”) usually includes, among other things, an infusion tube and a drip chamber and is used to supply the drug, which is present as infusion fluid and exits an infusion container (infusion bottle, infusion bag, etc.), to the patient access (cannula, venous catheter, etc.). An infusion set is therefore a system for administering drugs. The infusion fluid can be delivered by gravity and/or by means of an infusion pump. Optionally, the infusion set can also comprise a flow controller, for example in the form of a roller clamp, with which the speed of administration of the infusion fluid can be controlled.

Before a patient is administered an infusion, the infusion tube of the infusion set is filled with the infusion fluid and thus vented. This prevents air from entering the patient's bloodstream, where it could cause a dangerous air embolism. Filling the infusion tube before administering the infusion is commonly referred to as “venting” or “priming” the infusion tube. To prevent infusion fluid from leaking from the end of the infusion tube facing away from the infusion container or drip chamber during venting, closures are known in the prior art that retain infusion fluid in the infusion tube. These closures are known as “prime stop caps”, among other things.

Such closures can prevent the unintentional leakage of infusion fluid. However, they cannot prevent the air which is in the infusion tube before venting and is displaced by the infusion fluid in the course of venting from being released to the environment. Since the air released to the environment has been in contact with the infusion fluid, it may contain potentially harmful components in the form of gases or aerosols. Therefore, in practice, infusion devices are vented with non-hazardous flushing solutions. Only after the infusion device is connected to the patient is the CMR drug administered. However, this procedure is complicated, as it requires the implementation of additional steps and the use of different fluids.

### SUMMARY

It is therefore a task of the invention to simplify the reduction or prevention of the unintentional escape of gases and/or aerosols from an infusion arrangement. A further object of the invention is to provide an improved tube connection for the infusion tube, in particular a tube connector, with the aid of which an unintentional escape of gases and/or aerosols can be prevented or completely avoided in a simple manner. A further object of the invention is to provide improved methods for venting an infusion tube, in which the unintentional escape of gases and/or aerosols can be reduced or completely avoided.

The tube connector according to the invention is a tube connector for an infusion tube. The tube connector is connectable or connected to an infusion container. The tube connector has a first fluid opening which is connectable to an infusion tube or a drip chamber connected to an infusion tube to provide a fluid connection between the infusion container and the infusion tube. The tube connector has a second fluid opening connectable to the infusion tube to provide a fluid connection between the infusion container and the infusion tube.

The tube connector includes a blocking device configured to block the second fluid opening of the tube connector from fluid exiting the tube connector.

This prevents, for example, the exiting of fluid from an infusion container connected to the tube connector through the second fluid opening of the tube connector.

The blocking device is preferably a check valve that allows fluid to enter the tube connector through the second fluid opening above a certain minimum pressure (valve opening pressure), but does not allow fluid to exit the tube connector through the second fluid opening.

Alternatively, the blocking device can be configured as a shut-off valve that can switch between at least the following positions: In a first position, fluid can flow through the second fluid opening. In a second position, fluid cannot flow through the second fluid opening. By placing the shut-off valve in the first position or leaving it in the first position when fluid is to flow through the second fluid opening into the container port, and by placing the shut-off valve in the second position or leaving it in the second position when fluid is not to flow through the second fluid opening out of the container port, the shut-off valve provides the same function as a check valve.

The shut-off valve can be operated manually, for example, by a rotary actuation.

Alternatively, other elements can be used to provide this function, for example by another switchable valve, especially a manually switchable valve.

The use of a check valve offers the advantage that no additional step is required to open and close the valve. The use of a shut-off valve can be advantageous if a substance (e.g., drug bolus) is to be supplied to the infusion system through the second fluid opening, because then the supplied substance does not have to overcome the valve opening pressure of a check valve. The supply of a substance through the second fluid opening is also possible when using a check valve.

In addition to providing a fluid connection between an infusion tube and an infusion container via the first fluid opening, the tube connector according to the invention can provide another fluid connection between an infusion tube and an infusion container via the second fluid opening.

With the tube connector according to the invention, it is possible to connect the infusion tube to the infusion container during venting in such a way that the air which escapes from the infusion tube during venting is not released into the environment, but flows into the infusion container. This can reduce or completely prevent contamination of the environment with toxic substances contained in said air.

The tube connector according to the invention therefore offers the possibility of a closed system during venting.

In addition, the blocking device ensures that no fluid exits unintentionally from the tube connector when the second fluid opening is not connected to an infusion tube, without having to close it with a separate closure element for this purpose.

The tube connector according to the invention can have coupling devices of any type. Preferably, the coupling devices are for forming dry-locking connections, in particular needle-based dry-locking connections.

The infusion set according to the invention is an infusion set comprising a tube connector according to the invention and an infusion tube. The infusion set optionally comprises a drip chamber and/or a venous access.

The method according to the invention according to a first alternative is a method for venting an infusion tube comprising the steps:
A) connecting a first end of an infusion tube to an infusion container having an infusion fluid therein, such that there is a fluid connection between the infusion container and the first end of the infusion tube,
B) connecting a second end of the infusion tube to the infusion container so that there is a fluid connection between the infusion container and the second end of the infusion tube,
C) introducing infusion fluid into the infusion tube,
D) disconnecting the second end of the infusion tube from the infusion container after the infusion tube has been filled with infusion fluid in step C.

Steps A and B can be executed in any order. Steps A and B are both executed before step C.

The method according to the invention according to a further alternative is a method for venting an infusion tube comprising the steps:
A) connecting a first end of an infusion tube to an infusion container having an infusion fluid therein, such that there is a fluid connection between the infusion container and the first end of the infusion tube,
B) connecting a second end of the infusion tube to a collection vessel so that there is a fluid connection between the collection vessel and the second end of the infusion tube,
C) introducing infusion fluid into the infusion tube,
D) disconnecting the second end of the infusion tube from the collection vessel after the infusion tube has been filled with infusion fluid in step C.

Steps A and B can be executed in any order. Steps A and B are both executed before step C.

According to the methods of the invention, the infusion tube is connected to the infusion container or a collection vessel during the venting process in such a way that the air which escapes from the infusion tube during venting is not released into the environment, but flows into the infusion container or the collection vessel. In this way, contamination of the environment with toxic substances contained in said air can be reduced or avoided altogether without having to use a different fluid for venting than is used during administration of the infusion.

The methods according to the invention therefore create the advantageous situation of having a closed system during venting.

### BRIEF DESCRIPTION OF THE DRAWING FIGURES

Further features, expedients and advantages of the invention are described below by means of exemplary embodiments with reference to the attached drawing figures.

FIG. 1 shows a schematic, partially sectional view of an infusion arrangement comprising the tube connector according to a first embodiment of the invention, wherein the individual components of the infusion arrangement are shown disconnected from each other.

FIG. 2 shows a schematic, partially sectional view of the infusion arrangement, which comprises the tube connector according to the invention, according to the first embodiment, during venting of the infusion tube.

FIG. 3 shows a schematic, partially sectional view of a tube connector according to a second embodiment of the invention, which is connected to an infusion container.

FIG. 4 shows a schematic, partially sectional view of a tube connector according to the invention in accordance with a specific embodiment of the invention.

### DETAILED DESCRIPTION

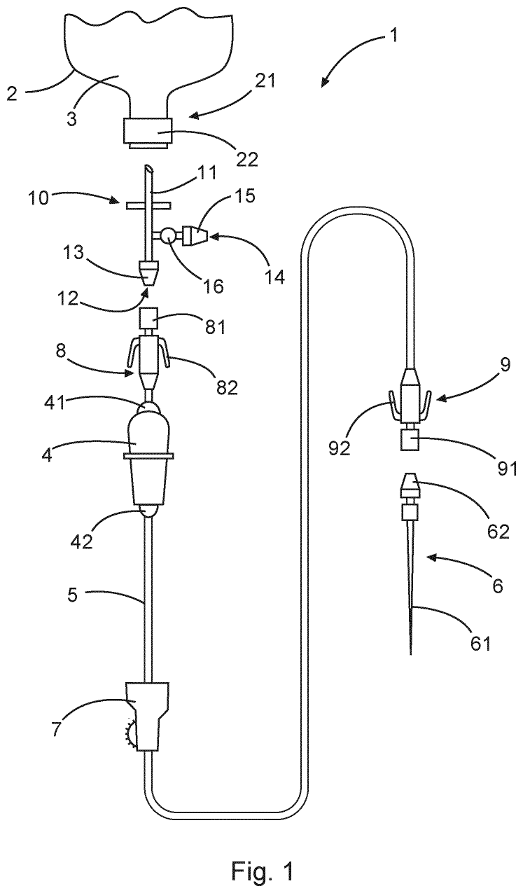

FIG. 1 shows a schematic view of an infusion arrangement 1 comprising the tube connector according to a first embodiment of the invention. For clarity, the individual components of the infusion arrangement 1 are shown disconnected from one another, i.e. in a disconnected state. The infusion arrangement 1 comprises the components described in more detail below, which serve to administer an infusion.

An infusion container 2, which is only partially shown, contains an infusion solution 3. The infusion container 2 is shown in an operating position in which the outlet region 21 or the closure 22 of the infusion container 2 faces downwards. The infusion container 2 can be a bottle or a bag, for example.

The infusion arrangement 1 further comprises a drip chamber 4, an infusion tube 5 and a patient access 6. The function of the drip chamber 4 is to receive the infusion fluid 3 exiting the infusion container 2 in a controlled manner and to deliver it to the infusion tube 5. For this purpose, the infusion tube 5 is connected at the first of its two ends to the outlet 42 of the drip chamber 4. The function of the infusion tube 5 is to supply the infusion fluid 3 exiting the drip chamber 4 to the patient access 6. The function of the patient access 6, which may be, for example, a venous cannula with a hollow needle 61 or a venous catheter with a hollow needle 61, is to allow the infusion fluid 3 exiting the infusion tube 5 to enter the patient's body, for example via a vein.

The infusion arrangement 1 further comprises a tube connector 10 according to a first embodiment of the invention.

The tube connector 10 can be connected to the infusion container 2. For this purpose, the tube connector 10 has a connecting structure 11. The connecting structure 11 is exemplarily shown as a hollow mandrel (so-called “spike”), with which a septum or a seal in the region of the closure 22 of the infusion container 2 can be pierced in order to establish a fluid connection with the interior of the infusion container 2. Alternatively, the connecting structure 11 may be in the form of a coupling device, a threaded connection or a push-fit connection. It is essential that the connecting structure 11 is formed such that a fluid connection with the interior of the infusion container 2 can be established. In this context, it can be advantageous if the fluid connection, once established, cannot be released again in order to avoid an unintentional separation of the tube connector 10 and the infusion container 2 and thus to prevent the unintentional leakage of infusion fluid 3.

The tube connector 10 further comprises a first fluid opening 12. The first fluid opening 12 and the inlet 41 of the drip chamber 4 are connectable to each other, so that a fluid connection between the infusion container 2 and the infusion tube 5 is provided via the first fluid opening 12. When, on the one hand, the infusion container 2 and the tube connector 10 are connected to each other and, on the other hand, the first fluid opening 12 and the inlet 41 of the drip chamber 4 are connected to each other, a fluid connection is provided between the infusion tube 5 and the infusion container 2 via the first fluid opening 12. As a result of this fluid connection, infusion fluid 3 can flow from the interior of the infusion container 2 into the infusion tube 5.

The outlet region 21 or closure 22 of the infusion container 2 faces downward in the operating position. Preferably, the first fluid opening 12 is arranged so that it also faces downward when connected to an infusion container in the operating position. In this case, the direction in which the first fluid opening 12 faces is more preferably inclined at most 10° with respect to the vertical, even more preferably at most 5° with respect to the vertical.

In order to be able to connect the first fluid opening 12 with the drip chamber 4 and thus with the first of the two ends of the infusion tube 5, the tube connector 10 has a first coupling device 13 in the region of the first fluid opening 12. The inlet 41 of the drip chamber 4 is connected to the second coupling device 8. The first coupling device 13 and the second coupling device 8 are complementary to each other, that is, the first coupling device 13 has a structure that can be brought into contact or engagement with the coupling element 81 of the second coupling device 8 such that there is a tight fluid connection between the first fluid opening 12 and the drip chamber 4. When the tube connector 10 is connected to an infusion container 2, there is then a fluid connection between the interior of the infusion container 2 and the infusion tube 5 such that infusion fluid 3 can enter the infusion tube 5 from the interior of the infusion container 2 via the drip chamber 4 via the first fluid opening 12.

It is possible that a connection between the first coupling device 13 and the second coupling device 8 is a releasable connection. It is preferred in this case that during and after the release of the connection between the first coupling device 13 and the second coupling device 8, the first coupling device 13 and the second coupling device 8 each are dry-locked. The coupling in this case is therefore a so-called break-lock coupling.

When the connection between the first coupling device 13 and the second coupling device 8 is a releasable connection, the first coupling device 13 and the second coupling device 8 preferably comprise a locking structure for locking the connection between the first and the second coupling device, wherein the locking is further preferably provided by an engagement and can be released by an operation of a closing mechanism 82.

The first coupling device 13 and the second coupling device 8 are of complementary types, for example the former of type A and the latter of type B. Coupling devices known in the prior art can be used, so that the tube connector 10 according to the invention can be used together with infusion tubes currently available on the market and thus does not require the use of further special components.

Alternatively, the connection between the first coupling device 13 and the second coupling device 8 may be a non-detachable connection. In this case, the connection cannot be disconnected again once it has been closed. Since, as a rule, both the infusion container 2 and the drip chamber 4 and the infusion tube 5 are disposed of together with the couplings and the tube connector 10 after an infusion has been administered, it is often not necessary to be able to disconnect the connection between the first coupling device 13 and the second coupling device 8 again.

In an alternative embodiment not shown in the drawing figures, the drip chamber is fixedly connected, for example welded or glued, to the first fluid opening of the tube connector.

The tube connector 10 further comprises a second fluid opening 14. The second fluid opening 14 and the second end of the infusion tube 5 are connectable to each other, so that a fluid connection between the infusion tube 5 and the infusion container 2 is provided via the second fluid opening 14. As a result of this fluid connection, a fluid from the infusion tube 5 can enter the interior of the infusion container 2.

The outlet region 21 or the closure 22 of the infusion container 2 faces downward in the operating position. Preferably, the second fluid opening 14 is configured to face in a lateral direction when connected to an infusion container in the operating position. Thereby, the direction in which the second fluid opening 14 faces is more preferably inclined at least 60° and/or at most 120° with respect to the vertical, even more preferably at least 80° and/or at most 100° with respect to the vertical.

In order to be able to connect the second fluid opening 14 with the second of the two ends of the infusion tube 5, the tube connector 10 comprises a third coupling device 15 in the region of the second fluid opening 14. The second end of the infusion tube 5 is connected to the fourth coupling device 9. The third coupling device 15 and the fourth coupling device 9 are complementary to each other, that is, the third coupling device 15 has a structure that can be brought into contact or engagement with the coupling element 91 of the fourth coupling device 9 such that there is a tight fluid connection between the second fluid opening 14 and the infusion tube 5. When the tube connector 10 is connected to an infusion container 2, there is then a fluid connection between the interior of the infusion container 2 and the infusion tube 5 such that a fluid can enter the interior of the infusion container 2 from the infusion tube 5 via the second fluid opening.

In this case, the third coupling device 15 and the fourth coupling device 9 are configured such that a connection between the third coupling device 15 and the fourth coupling device 9 is a releasable connection. While and after the connection between the third coupling device 15 and the fourth coupling device 9 is released, the third coupling device 15 dryly closes the second fluid opening 14 and the fourth coupling device 9 dryly closes the second end of the infusion tube 5, respectively. The coupling is therefore a so-called dry-break coupling.

The third coupling device 15 and the fourth coupling device 9 preferably comprise a locking structure for locking the connection between the third coupling device 15 and the fourth coupling device 9, wherein the locking is further preferably provided by snapping and can be released by an operation of a closing mechanism 92.

In order to be able to connect the patient access 6 to the second end of the infusion tube 5, the patient access has a fifth coupling device 62 in the region of one of its ends. The second end of the infusion tube 5 is connected to the fourth coupling device 9, as described above. The fourth coupling device 9 and the fifth coupling device 62 are complementary to each other, that is, the fifth coupling device 62 has a structure that can be brought into contact or engagement with the coupling element 91 of the fourth coupling device 9 such that there is a tight fluid connection between the patient access 6 and the infusion tube 5. When the tube connector 10 is connected to an infusion container 2, there is then a fluid connection between the interior of the infusion container 2 and the patient access 6 such that a fluid from the infusion container 2 can enter the patient access 6. When the patient access 6 is connected to the body of a patient, for example by the hollow needle 61 having been inserted into a vein of the body, there is then a fluid connection between the body and the interior of the infusion container 2 necessary for the administration of an infusion.

In this regard, the fourth coupling device 9 and the fifth coupling device 62 are preferably configured such that a connection between the fourth coupling device 9 and the fifth coupling device 62 is a releasable connection. When the connection between the fourth coupling device 9 and the fifth coupling device 62 is released, the fourth coupling device 9 dryly closes the second end of the infusion tube 5 and the fifth coupling device 62 dryly closes the patient access 6 in this case, respectively. The coupling in this case is therefore a so-called dry-break coupling.

The fourth coupling device 9 can thus optionally be connected either to the third coupling device 15 of the tube connector 10 or to the fifth coupling device 62 of the patient access 6. The connection between the fourth coupling device 9 and the third coupling device 15 is thereby present, for example, during and after venting and prior to administration of an infusion, as will be described below. The connection between the fourth coupling device 9 and the fifth coupling device 62 is thereby present, for example, during the administration of an infusion.

The third coupling device 15 and the fifth coupling device 62 are therefore coupling devices of the same type (for example, both of type A), while the fourth coupling device 9 is of the complementary type (for example, of type B).

If the first and second coupling devices 13, 8 also form a releasable coupling, they can either be of the same construction and size as the third, fourth and fifth coupling devices 15, 9, 62; this situation is illustrated in FIG. 1. This has the advantage that the infusion set has fewer different components overall. Alternatively, the first and second coupling devices 13, 8 may be of a construction and/or size different from the construction and/or size of the third, fourth and fifth coupling devices 15, 9, 62. This has the advantage that the infusion tube 5 cannot be mistakenly connected to the infusion container 2 upside down. The first and third coupling devices 13 and 15 can be of the same type; this situation is shown in FIG. 1. Alternatively, the first and third coupling devices 13 and 15 may be of complementary types.

The tube connector 10 further comprises a blocking device 16 in the region of the second fluid opening. In preferred embodiments of the invention, the blocking device 16 is a check valve. A check valve is understood to be any valve which allows a fluid to pass through the valve in one flow direction while not allowing, i.e. shutting off, the passage of a fluid in the opposite direction. For example, the check valve 16 may be a slotted cup valve or a poppet valve with a blocking effect for one flow direction or a check valve. As an alternative to a check valve, a shut-off valve or other switchable valve can be provided as a blocking device, for example.

A drip chamber 4 is not required as a component of the infusion arrangement 1 in order to be able to use the advantageous properties of the tube connector 10 according to the invention. Alternatively, the infusion tube 5 can also be connected directly to the tube connector 10, for example via coupling devices 13, 8 of the type described above.

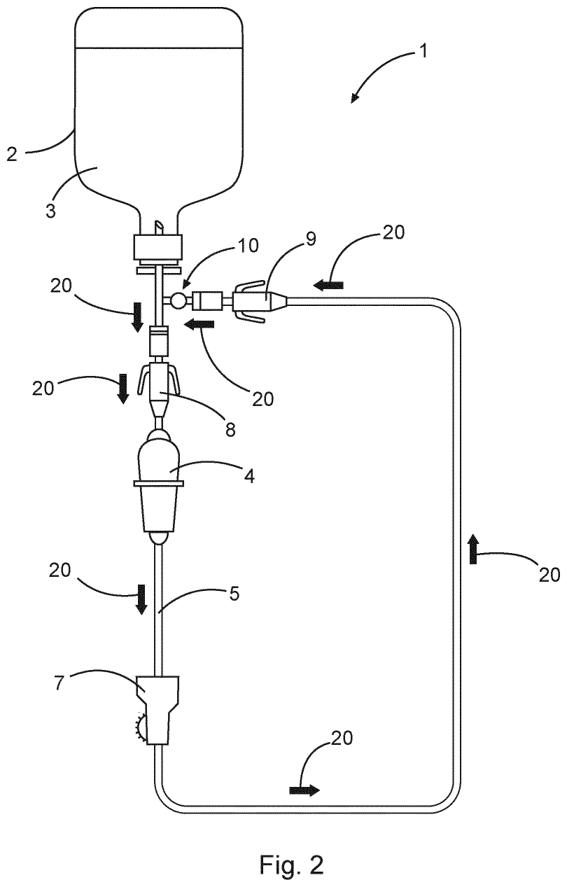

FIG. 2 shows a schematic view of the infusion arrangement 1, which comprises the tube connector 10 according to the invention in accordance with the first embodiment. The individual components of the arrangement are thereby connected to each other, i.e. connected, with the exception of the patient access 6 (not shown in FIG. 2).

In this case, the components are connected to each other in such a way that a closed state of the system is present in which the venting of the infusion tube 5 can be carried out without air escaping from the infusion tube being released into the environment.

Specifically, the first fluid opening 12 is connected to the drip chamber 4 and thus to the first end of the infusion tube 5. For this purpose, the first coupling device 13 and the second coupling device 8 are connected to each other so that there is a tight fluid connection between the first fluid opening 12 and the drip chamber 4. The tube connector 10 is connected to an infusion container 2. Thus, there is a fluid connection between the interior of the infusion container 2 and the infusion tube 5 so that infusion fluid 3 can enter the first end of the infusion tube 5 from the interior of the infusion container 2 via the drip chamber 4.

The second fluid opening 14 is connected to the second end of the infusion tube 5. For this purpose, the third coupling device 15 and the fourth coupling device 9 are connected to each other so that there is a tight fluid connection between the second fluid opening 14 and the infusion tube 5. The tube connector 10 is connected to an infusion container 2. Thus, there is a fluid connection between the interior of the infusion container 2 and the infusion tube 5 so that a fluid from the second end of the infusion tube 5 can enter the interior of the infusion container 2. The blocking device prevents a fluid from reversely entering the second end of the infusion tube 5 from the tube connector 10 or the interior of the infusion container 2. When a check valve is used as a blocking device 16, no additional measures are required to provide this function because a check valve is automatically permeable to fluid in one direction and blocks fluid passage in the opposite direction.

To vent the infusion tube 5, in which initially there is air, infusion fluid 3 is allowed to enter the infusion tube 5 from the infusion container 2 through the first fluid opening 12. In doing so, the infusion fluid 3 takes its path via the drip chamber 4, if a drip chamber 4 is present. If necessary, the entry of infusion fluid 3 into infusion tube 5 can be initiated, for example, by applying pressure to infusion container 2 or drip chamber 4 to start the outflow of infusion fluid 3. The infusion fluid 3 fills the infusion tube 5 and displaces the air present in the infusion tube 5. Said air enters the infusion container 2 via the second fluid opening 14 and therefore cannot escape into the environment. Therefore, no contamination of the environment by escaping air takes place. The blocking device prevents infusion fluid 3 from exiting the infusion container 2 through the second fluid opening 14. If the infusion arrangement 1 comprises a drip chamber 4, it may be necessary to first fill the drip chamber 4 with infusion fluid 3 before filling the infusion tube 5 with infusion fluid 3. This can be achieved, for example, by shutting off the infusion tube 5 at the beginning of the venting process until the drip chamber 4 is filled with infusion fluid 3. A roller clamp 7, for example, can be used for shutting off.

The flow direction of the infusion fluid 3 entering the infusion tube 5 in the course of venting and of the air escaping from the infusion tube 5 in the course of venting is indicated by arrows 20 in FIG. 2.

When venting has taken place, i.e. when the infusion tube 5 is filled with infusion fluid 3, the connection between the third coupling device 15 and the fourth coupling device 9 can be disconnected. Since these are dry-locking coupling devices, their outer surfaces are not wetted with infusion fluid, so there is no contamination of the environment as a result of the disconnection.

Subsequently, the fourth coupling device 9 can be connected to the fifth coupling device 62 of the patient access 6. This situation is not shown in the drawing figures.

Therefore, in order to perform the venting, it is possible to proceed according to the method according to the invention, in particular using the tube connector 10 according to the invention, which comprises the following steps:
A) connecting the first end of the infusion tube 5 to the infusion container 2, thereby establishing a fluid connection between the interior of the infusion container 2 and the first end of the infusion tube 5. For example, the first coupling device 13 and second coupling device 8, as described above, are used for this purpose.
B) connecting the second end of the infusion tube 5 to the infusion container 2, thereby establishing a fluid connection between the interior of the infusion container 2 and the second end of the infusion tube 5. For example, the third coupling device 15 and fourth coupling device 9, as described above, are used for this purpose.
C) introducing infusion fluid 3 into infusion tube 5 to fill infusion tube 5 with infusion fluid 3.
D) disconnecting the second end of the infusion tube 5 from the infusion container 2 after the infusion tube 5 has been filled with infusion fluid 3 in step C).

Steps A and B can be executed in any order. However, steps A and B are both executed before step C.

In an alternative method also according to the invention with the same steps A and C, the use of a special tube connector is not necessary. In step B, in the alternative method, the second end of the infusion tube 5 is not connected to the infusion container 2, but to a collection vessel. This establishes a fluid connection between the collection vessel and the second end of the infusion tube. For example, the third coupling device 15 and a complementary coupling device of the collection vessel serve for this purpose. Accordingly, in the alternative method, in step D, the second end of the infusion tube 5 is disconnected from the infusion container 2 after the infusion tube 5 has been filled with infusion fluid 3 in step C. In the alternative method, steps A and B can also be performed in any order. However, steps A and B are both performed before step C.

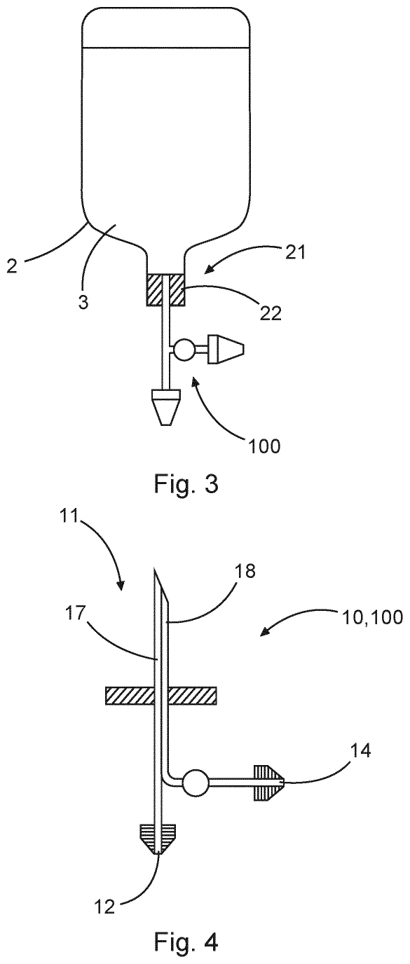

FIG. 3 shows a schematic, partially sectional view of a tube connector 100 according to a second embodiment of the invention, which is connected to an infusion container 2. The tube connector 100 according to the second embodiment differs from the tube connector 10 according to the first embodiment in that the first is directly connected to the closure 22 of the infusion container 2. The connection of the infusion container 2 and the tube connector 100 is non-detachable. For example, the direct connection of closure 22 and tube connector 100 can be achieved by welding or gluing these components together or by making them an integral component.

FIG. 4 shows in a schematic, partially sectional view of a tube connector 10, 100 according to the invention, how according to a specific embodiment of the invention the fluid channels run inside the tube connector 10, 100. A first fluid channel 17 provides the fluid connection between an infusion tube connected to the first fluid opening 12 (not shown in FIG. 4) or a drip chamber connected to an infusion tube (not shown in FIG. 4) and an infusion container connected to the tube connector (not shown in FIG. 4). The connecting structure 11 is shown here only as an example as a hollow mandrel, other suitable connecting structures or a direct connection to the closure of the infusion container 2 are equally possible. A second fluid channel 18 provides the fluid connection between an infusion tube (not shown in FIG. 4) connected to the second fluid opening 14 and an infusion container (not shown in FIG. 4) connected to the tube connector. The first fluid channel 17 and the second fluid channel 18 are separate from each other, that is, they each have a separate opening facing into the interior of an infusion container connected to the tube connector 10, 100.

In other embodiments, one of the fluid channels 17, 18 branches off from the other fluid channel.

The tube connector 10 according to the invention unfolds its advantageous properties not only in connection with the venting of an infusion tube. The tube connector 10 according to the invention also offers the possibility of introducing a substance into the infusion system through the second fluid opening 14 before or during the performance of an infusion, in order to administer it to the patient. The tube connector 10 according to the invention further provides the ability to connect a second infusion container to the second fluid opening 14 to administer an infusion fluid therein to the patient after administration of an infusion fluid from a first infusion container connected to the first fluid opening has been completed. In this regard, no venting is required prior to administration of the infusion solution from the second infusion container. Furthermore, it is not necessary to disconnect the first infusion container from the tube connector 10 before administration of the infusion solution from the second infusion container can begin. In this regard, it is advantageous if a check valve or other blocking device which shuts off the passage of fluid in one direction is arranged in the region of the first fluid opening 12, which prevents fluid from flowing back through the first fluid opening 12 into the first infusion container. It is possible to administer infusion solutions from the first and second infusion containers simultaneously using the same principle. It is further possible to provide the tube connector 10 with more than two fluid openings, so that infusion solutions from more than two infusion containers can be administered successively or simultaneously according to the same principle.

1. A tube connector for an infusion tube,
wherein the tube connector is connectable to an infusion container,
wherein the tube connector has a first fluid opening connectable to an infusion tube or a drip chamber connected to an infusion tube to provide a first fluid connection between the infusion container and the infusion tube,
wherein the tube connector has a second fluid opening connectable to the infusion tube to provide a second fluid connection between the infusion container and the infusion tube, and
wherein the tube connector comprises a blocking device configured to block the second fluid opening against a release of fluid from the tube connector.

2. The tube connector according to claim 1,
wherein the blocking device is configured as a check valve.

3. The tube connector according to claim 1,
wherein the tube connector has a connecting structure with which the tube connector is connectable to the infusion container.

4. The tube connector according to claim 1,
wherein the tube connector comprises a first fluid channel that provides the first fluid connection between the infusion container and the infusion tube,
wherein the tube connector comprises a second fluid channel that provides the second fluid connection between the infusion container and the infusion tube, and
wherein the first fluid channel and the second fluid channel are formed separately from each other.

5. The tube connector according to claim 1,
wherein the first fluid opening faces downward when the tube connector is connected to the infusion container arranged in an operating position, and
wherein the second fluid opening faces in a lateral direction when the tube connector is connected to the infusion container arranged in the operating position.

6. The tube connector according to claim 1,
wherein the tube connector has, in a region of the first fluid opening, a first coupling device which is connectable to a second coupling device, which is complementary to the first coupling device and attached to a first end of the infusion tube or to an inlet of the drip chamber connected to the first end of the infusion tube, in order to provide the first fluid connection between the infusion container and the infusion tube.

7. The tube connector according to claim 6,
wherein the first coupling device and the second coupling device provide a needle-based coupling.

8. The tube connector according to claim 6,
wherein the first coupling device and the second coupling device comprise a locking structure for locking a connection between the first coupling device and the second coupling device.

9. The tube connector according to claim 1,
wherein the first fluid opening is non-detachably connectable to a first end of the infusion tube or to an inlet of a drip chamber connected to the first end of the infusion tube.

10. The tube connector according to claim 1,
wherein the tube connector has, in a region of the second fluid opening, a third coupling device which is connectable to a fourth coupling device, which is complementary to the third coupling device and attached to a second end of the infusion tube, in order to provide the second fluid connection between the infusion container and the infusion tube,
wherein a connection between the third coupling device and the fourth coupling device is a releasable connection, and
wherein the third coupling device and the fourth coupling device are dry-locked during and after disconnection of the third coupling device from the fourth coupling device.

11. The tube connector according to claim 10,
wherein the third coupling device and the fourth coupling device provide a needle-based coupling.

12. The tube connector according to claim 10,
wherein the third coupling device and the fourth coupling device have a locking structure for locking a connection between the third coupling device and the fourth coupling device.

13. An infusion set comprising:
a tube connector according to claim 1; and
an infusion tube.

14. A method for venting an infusion tube comprising the steps of:
A) connecting a first end of an infusion tube to an infusion container containing an infusion fluid, such that there is a first fluid connection between the infusion container and the first end of the infusion tube;
B) connecting a second end of the infusion tube to the infusion container so that there is a second fluid connection between the infusion container and the second end of the infusion tube;
C) introducing the infusion fluid into the infusion tube; and
D) disconnecting the second end of the infusion tube from the infusion container after the infusion tube has been filled with the infusion fluid in step C,
wherein steps A and B are carried out in any order, and
wherein steps A and B are both executed before step C.

15. A method for venting an infusion tube comprising the steps of:
A) connecting a first end of an infusion tube to an infusion container containing an infusion fluid, such that there is a first fluid connection between the infusion container and the first end of the infusion tube;
B) connecting a second end of the infusion tube to a collection vessel so that there is a second fluid connection between the collection vessel and the second end of the infusion tube;
C) introducing the infusion fluid into the infusion tube; and
D) disconnecting the second end of the infusion tube from the collection vessel after the infusion tube has been filled with the infusion fluid in step C,
wherein steps A and B are carried out in any order, and
wherein steps A and B are both executed before step C.
